(19) United States
(12) Patent Application Publication
(10) Pub. No.: US 2007/0203367 A1
(43) Pub. Date: Aug. 30, 2007

(54) POLYETHERPOLYOL HAVING STEREOREGULARITY AND METHOD OF PREPARING THE SAME

(75) Inventors: Il Kim, Busan (KR); Sang Hyun Lee, Ulsan (KR); Jun Tai An, Ulsan (KR)

Correspondence Address: EGBERT LAW OFFICES, 412 MAIN STREET, 7TH FLOOR, HOUSTON, TX, 77002, US

(73) Assignee: SKC Inc., Ulsan (KR)

(21) Appl. No.: 11/741,080

(22) Filed: Apr. 27, 2007

Related U.S. Application Data
(62) Division of application No. 10/828,800, filed on Apr. 22, 2004.

(30) Foreign Application Priority Data
Feb. 3, 2004 (KR) 2004-7102

(51) Int. Cl. C07C 41/03

(52) U.S. Cl. 568/679

(57) ABSTRACT

A polyetherpolyol for use in production of polyurethane includes polyetherpolyol carbons having stereoregularity of syndiotactic triad. Further, a method of preparing such polyetherpolyol is provided. The polyurethane resulting from stereoregular polyetherpolyol is superior in physical properties to polyuretanes obtained by use of atactic polyetherpolyol.

### CROSS-REFERENCE TO RELATED U.S. APPLICATIONS

Not applicable.

### STATEMENT REGARDING FEDERALLY SPONSORED RESEARCH OR DEVELOPMENT

Not applicable.

### NAMES OF PARTIES TO A JOINT RESEARCH AGREEMENT

Not applicable.

### REFERENCE TO AN APPENDIX SUBMITTED ON COMPACT DISC

Not applicable.

### BACKGROUND OF THE INVENTION

1. Field of the Invention

The present invention relates to stereoregular polyetherpolyol and a production method thereof. In polyetherpolyol, which is used together with isocyanate as a production material of polyurethane, carbons connected to oxygen atoms between neighboring ether bonds are present in the forms of methine, methylene and methyl, in which methine carbon has a chiral structure.

It is generally known that polyurethane resulting from atactic polyol prepared in the presence of a basic catalyst is inferior in physical properties including elasticity, tensile strength, elongation, etc., to polyurethanes obtained by using stereoregular polyol.

Therefore, the present invention is directed to polyetherpolyol having stereoregularity as spatial sequences of constitutive carbons thereof, which is suitable for use in production of polyurethane having excellent mechanical properties, such as tensile strength, elongation and elasticity, and a method of preparing the same.

2. Description of Related Art Including Information Disclosed Under 37 CFR 1.97 and 37 CFR 1.98

Typically used for preparation of polyol, a starter is exemplified by materials having polyvalent hydroxide groups, such as propylene glycol, ethylene glycol, glycerine, methyl glucoside, glucose, trimethylol propane, pentaerythritol, ethylene diamine, toluene diamine, sugar, sorbitol, etc. In addition, examples of a monomer to be polymerized include compounds having epoxy groups (epoxy compound), such as ethylene oxide, propylene oxide, butylene oxide, etc. Further, a catalyst includes a basic catalyst, for example, sodium hydroxide, potassium hydroxide, cesium oxide, trimethyl amine, triethyl amine, etc., and a metal catalyst, for example, AlCl₃, ZnCl₂, FeCl₃, BF₃, BCl₃, BeCl₂, FeBr₃, SnCl₄, TiCl₄, ZrCl₄, ZnCl₂, etc. Also, there is used an organic metal catalyst, including Al(OR)₃, Mg(OR)₂, Zn(OR)₂, etc., for use in epoxide polymerization (ring-opening reaction of an epoxy group), in which R means an alkyl group.

Polyol prepared in the presence of the basic catalyst has unsaturated polyol by a side-reaction upon epoxide polymerization, with a molecular weight less than 6000. Hence, such polyol cannot be used to produce polyurethane having high elasticity. Although an amine base useful as the basic catalyst is disadvantageous in terms of difficulty in removal thereof and offensive odor due to a residue thereof, it may be partially applied to produce rigid polyurethane. On the other hand, since the metal catalyst, such as AlCl₃, ZnCl₂, FeCl₃, and BF₃, hardly causes the side-reaction of monol as the disadvantage of the basic catalyst, it may be employed to prepare polyol having higher molecular weight. However, the metal catalyst suffers from use of larger amounts, attributed to low reactivity of propylene oxide thereto, and difficulty in handling.

To decrease the concentration of the unsaturated polyol caused by the side-reaction of the epoxide polymerization, there is proposed use of a CsOH catalyst or a double metal cyanide catalyst, instead of the basic catalyst. The CsOH catalyst can reduce the degree of unsaturation by about 50%, compared to conventional catalysts. The double metal cyanide catalyst can minimize the side-reaction of monol, thus preparing almost pure polyol having 0.01 meq/9 or less of monol and polymer products having higher molecular weights, such as polyether, polyester and polyetherester polyol. The double metal cyanide catalyst, which is prepared by reacting a metal salt, a metal cyanide salt and a complexing agent, has a general formula of Mₐ[M′(CN)₆]_{b}L_{c} in which M represents Zn (II), Fe (II), Ni (II), Mn (II), Co (II), Sn (II), Pb (II), Fe (III), Mo (IV), Mo (VI), Al (II), V (V), V (IV), Sr (II), W (IV), W (VI), Cu (II), and Cr (III). The metal salt has a general formula Of M(X)ₙ, in which X shows an anion selected from among halide, hydroxide, sulfate, carbonate, cyanide, oxalate, thiocyanate, isocyanate, isothiocyanate, carboxylate, and nitrate, and n is 1-3 and satisfies a valence of M. The metal salt is exemplified by zinc chloride, zinc bromide, zinc acetate, zinc acetonylacetonate, zinc benzoate, zinc nitrate, iron (II) bromide, cobalt (II) chloride, cobalt (II) thiocyanate, nickel (II) formate, and nickel (II) nitrate. The metal cyanide salt (M′) has a general formula of (Y)aM′C(N)b(A)c, in which M′ is selected from among Fe (II), Fe (III), Co (II), Co (III), Cr (II), Cr(III), Mn (II), Mn (III), Ir (III), Ni (II), Rh (III), Ru (II), V (V), and V (IV), and Y is an alkali metal ion or alkaline metal ion, and A is an ion selected from among halide, hydroxide, sulfate, carbonate, cyanide, oxalate, thiocyanate, isocyanate, isothiocyanate, carboxylate, and nitrate and a and b are integers larger than 1, and a sum of a, b and c is equal to that of electrovalences of M and M′. The cyanide salt is exemplified by potassium hexacyanocobaltate (III), potassium hexacyanoferrate (II), potassium hexacyanoferrate (III), calcium hexacyanocobaltate (II), and lithium hexacyanoferrate (II). As for the complexing agent (L), U.S. Pat. Nos. 4,477,589, 3,821,505, and 5,158,922 disclose use of an organic complexing agent, such as ethyleneglycol or dimethylether which thus results in preparation of a catalyst having epoxide reactivity of 0.1-0.5 kg-PO/g-Co/min. Further, U.S. Pat. No. 5,158,922 discloses use of alcohol, aldehyde, ketone, ether, ester, amide, urea, and nitrile, and U.S. Pat. No. 5,780,584 discloses use of tert-butyl alcohol as the complexing agent, therefore resulting in preparation of polyol having 0.01 meq/g or less of unsaturated polyol. In addition, U.S. Pat. Nos. 5,482,908 and 5,789,626 disclose polyether for use in preparation of a catalyst. However, catalysts resulting from use of the above mentioned complexing agent have problems, including very long activation time of 2-4 hours, upon polymerization of epoxide such as propylene oxide. Moreover, a solvent used for preparation of the double metal catalyst should be removed by means of an expensive raw material, thus negating economic benefits and reducing productivity.

Meanwhile, polyetherpolyol resulting from propylene oxide through a ring-opening reaction of epoxide, according to reaction conditions, such as types of catalysts and solvents, has a head-to-tail sequence, a head-to-head sequence and a random sequence, as represented by the following formulas: 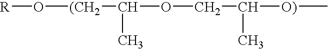
Head-to-Tail Sequence; 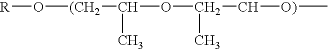
Head-to-Head Sequence; 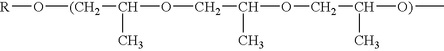
Random Sequence.

Polyol commercially prepared by use of the basic catalyst includes 90% or more of head-to-tail sequence, and about 5% of head-to-head and about 5% of tail-to-tail sequences. Polyol has —OH terminal groups with a ratio of primary alcohol/secondary alcohol of 1/9 or more, due to 90% or more of head-to-tail sequence present in polyol, and thus, exhibits low reactivity upon production of polyurethane. Accordingly, in polyurethane fields requiring high reactivity, such as automotive seats, ethylene oxide may be added to the terminal of polyol.

Chiral carbon of polyol resulting from propylene oxide has two optical structures, that is, isotactic and syndiotactic structures. Hence, polyol having stereoregularity includes optical properties as well as crystalline polymer morphology, which can function to increase physical properties of polyurethane. Polyol obtained by use of propylene oxide has three carbons between oxygen atoms of neighboring ether bonds, for example, methine carbon of —CH═, methylene carbon of —CH₂—, and methyl carbon of —CH₃—In C-NMR, the methylene carbon peaks at 72.4-73.0 ppm, and the methyl carbon peaks at 16.8-17.0 ppm, and the methine carbon peaks at 74.6-75.0 ppm. Referring to FIG. 1, the methylene carbon has two peaks at 72-73 ppm, which is referred to as a diad stereoregularity. In addition, three peaks of the methine carbon at 74.6-75.0 ppm are referred to as a triad stereoregularity. Even though the methylene carbon has an atactic structure with a meso structure at 72.85 ppm and a racemic structure at 72.62 and 72.46 ppm, the methine carbon has syndiotactic stereoregularity.

The methine carbon at 74.6-75.0 ppm of C-NMR has stereoregularity of syndiotactic triad, with mm (meso+meso) at 74.95 ppm, mr+rm (meso racemic+meso racemic) at 74.79 ppm, and rr (racemic+racemic) at 74.62 ppm. Generally, as for polyol prepared by use of the basic catalyst, 60-70 mol % of mm and mr+rm carbons thereof have an atactic stereosequence. Reported in J. Am. Chem. Soc, 78, 1956, (C. C. Price), a stereoregular polymer, resulting from 1-propylene oxide and FeCl₃ catalyst, is disadvantageous in terms of use of expensive propylene oxide having optical properties, and low reactivity of propylene oxide by FeCl₃.

### BRIEF SUMMARY OF THE INVENTION

Leading to the present invention, the intensive and thorough research on polyetherpolyols, carried out by the present inventors aiming to avoid the problems encountered in the related art, resulted in the finding that an epoxy compound is subjected to epoxide polymerization under predetermined reaction conditions in the presence of a double metal cyanide complex catalyst including a complexing agent of primary alcohol and polyvalent alcohol, thereby obtaining polyetherpolyol with stereoregularity.

Accordingly, an object of the present invention is to provide polyetherpolyol in which constitutive carbons have stereoregularity of syndiotactic triad.

Another object of the present invention is to provide a method of preparing such polyetherpolyol.

### BRIEF DESCRIPTION OF THE SEVERAL VIEWS OF THE DRAWINGS

The above and other objects, features and other advantages of the present invention will be more clearly understood from the following detailed description taken in conjunction with the accompanying drawing.

FIG. 1 is a graphic illustration of a C-NMR spectrum showing two peaks of methylene carbon at 72 and 73 ppm.

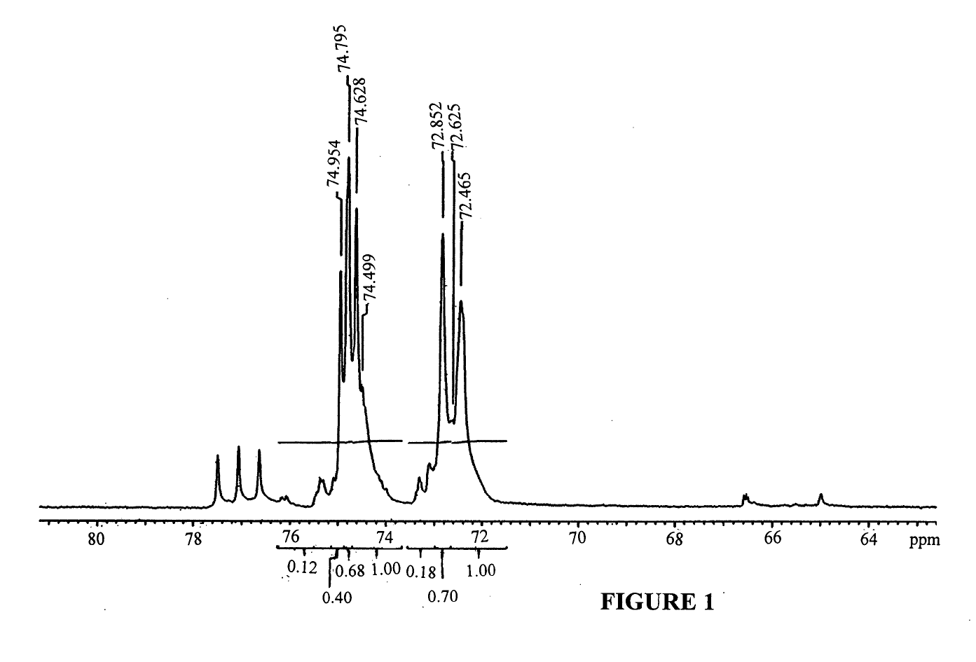

### DETAILED DESCRIPTION OF THE INVENTION

Based on the present invention, stereoregular polyetherpolyol, which is prepared through epoxide polymerization of propylene oxide in the presence of a double metal cyanide complex catalyst, has 45 mol % or more of methine carbon, with 95% or more of a head-to-tail sequence, and thus, can be used to produce polyurethane having high tensile strength and elasticity.

Proposed by the present inventors, a double metal cyanide complex catalyst for use in preparation of polyoxyetherpolyol or polyetherpolyol through epoxide polymerization has advantages, such as small production of unsaturated polyol, short catalyst activation time, and no need to remove catalyst remaining from polyol after the polymerization, and is represented by Formula 1, below (U.S. Pat. No. 6,627,575):
Mₐ[M′(CN)₆]_{b}L_{c}L′_{d} Formula 1
wherein, M and M′ are metal elements, and L and L′ are complexing agents, and a, b, c and d are integers, and a sum of a, b, c and d is equal to that of electrovalences of M and M′.

As for the double metal cyanide complex catalyst composed of a metal salt, a metal cyanide salt and a complexing agent, the complexing agent includes a monovalent alcohol (L) and a polyvalent alcohol (L′) together in the present invention although having been conventionally used in a single-type. Thus, the double metal cyanide complex catalyst of the present invention is characterized by containing about 5-80 wt % of monovalent alcohol and about 0.1-20 wt % of polyvalent alcohol.

As mentioned above, U.S. Pat. No. 6,627,575 discloses the advantages of the catalyst, such as small production of unsaturated polyol, short catalyst activation time, and no need to remove catalyst remaining from polyol after the polymerization. However, in the above patent, there is no found stereoregularity of polyol prepared in the presence of the above catalyst.

In the present invention, the monovalent alcohol is exemplified by tert-butanol, and the polyvalent alcohol includes polytetrahydrofuran.

As in U.S. Pat. Nos. 4,477,589, 3,829,505 and 5,158,922, to prepare the catalyst of the present invention, an aqueous solution of a metal salt, such as an excessive amount of zinc chloride, and a metal cyanide salt, such as potassium hexacyanocobaltate, are mixed with a complexing agent, such as tert-butanol, thereby obtaining a catalyst slurry containing a reaction product of the metal salt and the metal cyanide salt. Subsequently, the above catalyst slurry is mixed with polytetrahydrofuran (hereinafter, referred to as ‘poly(THF)’), to give a solid catalyst containing poly(THF), which is then separated from the catalyst slurry by means of a filtering process of a centrifuging process. The separated solid catalyst containing poly(THF) is washed with an aqueous solution of an organic complexing agent. That is, immediately after the solid catalyst is separated, it is washed with the organic complexing agent-containing aqueous solution. As such, the organic complexing agent in the aqueous solution is used in the amount of about 40-70 wt %. Further, a small amount (0.1-10 wt %) of poly(THF) is added to the washing solution, whereby the amount of poly(THF) in the catalyst can increase, thus further enhancing the activity of the catalyst. After the catalyst is washed, it is dried at 40-90° C. under vacuum of 26-30 in·Hg until the catalyst has a constantly maintained weight. Along with the resultant double metal catalyst, 100-150 g of polyetherpolyol having a molecular weight of 400-1000 is introduced into a reactor, and then heated at 80-130° C., and preferably, 95-112° C., followed by a dehydrating process for 1-3 hours under vacuum to remove a very small amount of remaining water from polyetherpolyol. When polyetherpolyol contains water of 1000 ppm or less, and particularly, 500 ppm or less, it is added with propylene oxide until pressure of the reactor is increased from 0.5 kgf to 3 kgf, and then, addition of propylene oxide is stopped. Propylene oxide is further added into the reactor at the point where the pressure of the reactor is reduced again to vacuum by the activation of the double metal catalyst. Thereby, the pressure of the reactor is maintained at a predetermined level. Thereafter, the reaction additionally takes place for 30 min to 1 hour to complete the reaction of propylene oxide. After the reaction is completed, propylene oxide residue is removed from the reaction product under reduced pressure. To analyze a stereosequence of methine carbon of carbons between neighboring ether bonds in polyol, an triad peak area of methine carbon at 74.6-75.0 ppm, of C-NMR is determined by means of an integral method, thus calculating a mol ratio of mm/mr/rr.

As such, m shows meso, and r shows racemic.

A better understanding of the present invention may be obtained through the following examples which are set forth to illustrate, but are not to be construed as the limit of the present invention.

### PREPARATIVE EXAMPLE 1

Preparation of Catalyst

Into a first beaker, 63 g of zinc chloride, 231 mL of distilled water and 42 mL of tert-butanol were placed and then mixed together (a mixture solution 1). Separately, 6.3 g of potassium hexacyanocobaltate was dissolved in 84 mL of distilled water in a second beaker (a mixture solution 2). The mixture solution 2 was added droplets to the mixture solution 1 at 50° C. for 1 hour while performing a stirring process by use of a mechanical stirrer. Thereafter, the reaction taken place for 10 min. A resultant solid was separated using a high-speed centrifuge, to obtain a catalyst slurry, which was then added with 10 mL of distilled water, 2 mL of tert-butanol and 20 g of poly(THF) (molecular weight: 1800). A solid catalyst component in the slurry was separated from the slurry using a high-speed centrifuge, after which the resulting wet catalyst cake was dried at 60° C. under vacuum of 30 in.Hg so as to have a predetermined weight, thereby producing a desired double metal catalyst 1.

### PREPARATIVE EXAMPLE 2

Preparation of Catalyst

Into a first beaker, 63 g of zinc chloride, 231 mL of distilled water and 42 mL of tert-butanol were placed and then mixed together (a mixture solution 1). Separately, 6.3 g of potassium hexacyanocobaltate was dissolved in 84 mL of distilled water in a second beaker (a mixture solution 2). The mixture solution 2 was added droplets to the mixture solution 1 at 50° C. for 1 hour while performing a stirring process by use of a mechanical stirrer. Thereafter, the reaction taken place for 10 min, and a resultant solid was separated using a high-speed centrifuge. Thereby, obtained catalyst slurry was added with 10 mL of distilled water, 2 mL of tert-butanol and 20 g of polyether polyol, from which a solid catalyst component was separated using a high-speed centrifuge. Subsequently, wet catalyst cake was dried at 60° C. under vacuum of 30 in.Hg so as to have a predetermined weight, thereby obtaining a desired double metal catalyst 2.

### EXAMPLE 1

In a 1 L high-pressure reactor, 122 g of polyether glycerin having a molecular weight of 550 as a starter polyol was mixed with 0.03 g of the double metal catalyst 1. This mixture was heated to 112° C. while being stirred, from which a very small amount of water was removed under reduced pressure. The addition of a propylene oxide monomer into the reactor was continued until pressure in the reactor was increased from vacuum to 0.5 kgf, and was then stopped. Subsequently, 592 g of propylene oxide was introduced at 1 g/min into the reactor at the point where the pressure of the reactor was reduced again to vacuum by the activation of the double metal catalyst, while a temperature of the reactor was controlled. Thereby, the pressure of the reactor was maintained at a predetermined level. After completion of the reaction, the reactor was allowed to stand at 112° C. for 30 min under vacuum to remove unreactant propylene oxide monomer from a reaction product.

Thus, obtained polyol was analyzed with C-NMR to confirm a mol ratio of methine carbon. The results are shown in Table 1, below.

### EXAMPLE 2

In a 1 L high-pressure reactor, 142 g of polyether glycol having a molecular weight of 400 as a starter was mixed with 0.03 g of the double metal catalyst 1. While this mixture was stirred, it was heated to 112° C., from which a very small amount of water was removed under reduced pressure. The addition of a propylene oxide monomer into the reactor was continued until pressure in the reactor was increased from vacuum to 0.5 kgf, and was then stopped. The double metal catalyst was activated in the reactor, and thus, the pressure of the reactor was reduced again to vacuum. At this time, 573 g of propylene oxide was introduced at 1 g/min into the reactor while a temperature of the reactor is controlled, so that the pressure of the reactor was maintained at a predetermined level. After completion of the reaction, the reactor was allowed to stand at 112° C. for 30 min under vacuum to remove unreactant propylene oxide from a reaction product.

Thus, obtained polyol was analyzed with C-NMR to confirm a mol ratio of methine carbon. The results are shown in Table 1, below.

TABLE 1

| | Reaction Condition | | | | | |
|---|---|---|---|---|---|---|
| | | Number of | | | | |
| | | Functional | Molecular | Mol %* | | |
| Ex. No. | Catalyst | Groups | Weight | mm | mr + rm | rr |
| 1 | Catalyst 1 | 3 | 3000 | 19.7 | 31.0 | 49.3 |
| 2 | Catalyst 1 | 2 | 2000 | 18.2 | 30.6 | 51.2 |

*C-NMR Peak Area % (mm = 74.95, mr + rm = 74.79, rr = 74.62)

### EXAMPLE 3

In a 1 L high-pressure reactor, 104 g of polyether glycerin having a molecular weight of 900 as a starter polyol was mixed with 0.032 g of the double metal catalyst 1. This mixture was heated to 112° C. while being stirred, from which a very small amount of water was removed under reduced pressure. The addition of a propylene oxide monomer into the reactor was continued until pressure in the reactor was increased from vacuum to 0.5 kgf, and was then stopped. The double metal catalyst was activated in the reactor, whereby the pressure of the reactor was reduced again to vacuum. In this case, 608 g of propylene oxide was introduced at 1 g/min into the reactor while a temperature of the reactor was controlled, so that the pressure of the reactor was maintained at a predetermined level. After completion of the reaction, the reactor was allowed to stand at 112° C. for 30 min under vacuum to remove unreactant propylene oxide from a reaction product. Thus, obtained polyol was analyzed with C-NMR to confirm a mol ratio of methine carbon. The results are given in Table 2, below.

### EXAMPLE 4

In a 1 L high-pressure reactor, 71 g of polyether glycol having a molecular weight of 400 as a starter was mixed with 0.03 g of the double metal catalyst 1. This mixture was heated to 112° C. while being stirred, from which a very small amount of water was removed under reduced pressure. The addition of a propylene oxide monomer into the reactor was continued until pressure in the reactor was increased from vacuum to 0.5 kgf, and was then stopped. When the pressure of the reactor was reduced again to vacuum by the activation of the double metal catalyst, addition of 643 g of propylene oxide at 1 g/min into the reactor was performed along with control of a temperature of the reactor. Thereby, the pressure of the reactor was maintained at a predetermined level. After completion of the reaction, the reactor was allowed to stand at 112° C. for 30 min under vacuum to remove unreactant propylene oxide from a reaction product. Thus, obtained polyol was analyzed with C-NMR to confirm a mol ratio methine carbon. The results are given in Table 2, below.

TABLE 2

| | Reaction Condition | | | | | |
|---|---|---|---|---|---|---|
| | | Number of | | | | |
| | | Functional | Molecular | Mol %* | | |
| Ex. No. | Catalyst | Groups | Weight | mm | mr + rm | rr |
| 3 | Catalyst 1 | 3 | 6000 | 19.5 | 30.8 | 49.7 |
| 4 | Catalyst 1 | 2 | 4000 | 18.8 | 31.2 | 50.0 |

### EXAMPLE 5

In a 1 L high-pressure reactor, 140 g of polypropylene sorbitol having a molecular weight of 600 as a starter was mixed with 0.03 g of the double metal catalyst 1. This mixture was heated to 112° C. while being stirred, from which a very small amount of water was removed under reduced pressure. The addition of a propylene oxide monomer into the reactor was continued until pressure in the reactor was increased from vacuum to 0.5 kgf, and was then stopped. Subsequently, 574 g of propylene oxide was introduced at 1 g/min into the reactor at the point where the pressure of the reactor was reduced again to vacuum by the activation of the double metal catalyst, while a temperature of the reactor was controlled. Thereby, the pressure of the reactor was maintained at a predetermined level. After completion of the reaction, the reactor was allowed to stand at 112° C. for 30 min under vacuum to remove unreactant propylene oxide from a reaction product. Thus, obtained polyol was analyzed with C-NMR to confirm a mol ratio of methine carbon. The results are represented in Table 3, below.

### EXAMPLE 6

Desired polyol was prepared in the same manner as in Example 5, with the exception that 140 g of polypropylene sorbitol having a molecular weight of 2400 as a starter and 0.03 g of the double metal catalyst 1 were mixed together, and 574 g of propylene oxide was used. The resultant polyol was analyzed with C-NMR to confirm a mol ratio of methine carbon. The results are represented in Table 3, below.

TABLE 3

Reaction Condition

| | Number of | | | | |
|---|---|---|---|---|---|
| | Functional | Molecular | Mol %* | | |
| Ex. No. | Groups | Weight | mm | mr + rm | rr |
| 5 | 6 | 3000 | 20.2 | 31.0 | 48.8 |
| 6 | 8 | 3000 | 19.4 | 30.6 | 50.0 |

### EXAMPLE 7

In a 1 L high-pressure reactor, 128 g of polyether glycerin having a molecular weight of 550 as a starter polyol was mixed with 0.03 g of the double metal catalyst 2. This mixture was heated to 112° C. while being stirred, from which a very small amount of water was removed under reduced pressure. The addition of a propylene oxide monomer into the reactor was continued until pressure in the reactor was increased from vacuum to 0.5 kgf, and was then stopped. Subsequently, 586 g of propylene oxide was introduced at 1 g/min into the reactor at the point where the pressure of the reactor was reduced again to vacuum due to the activation of the double metal catalyst, while a temperature of the reactor was controlled. Thereby, the pressure of the reactor was maintained at a predetermined level. After completion of the reaction, the reactor was allowed to stand at 112° C. for 30 min under vacuum to remove unreactant propylene oxide monomer from a reaction product. Thus, obtained polyol was analyzed with C-NMR to confirm a mol ratio of methine carbon. The results are shown in Table 4, below.

### EXAMPLE 8

In a 1 L high-pressure reactor, 142 g of polyether glycol having a molecular weight of 400 as a starter was mixed with 0.03 g of the double metal catalyst 2. While this mixture was stirred, it was heated to 112° C., from which a very small amount of water was removed under reduced pressure. The addition of a propylene oxide monomer into the reactor was continued until pressure in the reactor was increased from vacuum to 0.5 kgf, and was then stopped. The double metal catalyst was activated in the reactor, whereby the pressure of the reactor was reduced again to vacuum. At this time, 573 g of propylene oxide was introduced at 1 g/min into the reactor while a temperature of the reactor was controlled, so that the pressure of the reactor was maintained at a predetermined level. After completion of the reaction, the reactor was allowed to stand at 112° C. for 30 min under vacuum to remove unreactant propylene oxide from a reaction product. Thus, obtained polyol was analyzed with C-NMR to confirm a mol ratio of methine carbon. The results are shown in Table 4, below.

TABLE 4

Reaction Condition

| | | Number of | | | |
|---|---|---|---|---|---|
| | | Functional | Mol %* | | |
| Ex. No. | Catalyst | Groups | mm | mr + rm | rr |
| 7 | Catalyst 2 | 3 | 25.9 | 29.3 | 44.8 |
| 8 | Catalyst 2 | 2 | 26.7 | 30.5 | 42.8 |

### COMPARATIVE EXAMPLE 1

As a starter polyol, 128 g of polyether glycerin having a molecular weight of 550 was placed into a 1 L high-pressure reactor, to which 0.52 g of 98% KOH catalyst was added. This mixture was heated to 112° C. while being stirred, from which a very small amount of water was removed under reduced pressure. Subsequently, the addition of a propylene oxide monomer into the reactor was continued until pressure in the reactor was increased from vacuum to 0.5 kgf, and was then stopped. The double metal catalyst was activated in the reactor, whereby the pressure of the reactor was reduced again to vacuum. In this case, 586 g of propylene oxide was added at 1 g/min into the reactor while a temperature of the reactor was controlled, so that the pressure of the reactor was maintained at a predetermined level. After completion of the reaction, the reactor was allowed to stand at 112° C. for 30 min under vacuum to remove unreactant propylene oxide from a reaction product.

Thus, obtained polyol was analyzed with C-NMR to confirm a mol ratio of methine carbon. The results are given in Table 5, below.

### COMPARATIVE EXAMPLE 2

142 g of polyether glycol having a molecular weight of 400 as a starter was added into a 1 L high-pressure reactor, along with 0.52 g of 98% KOH catalyst. While this mixture was stirred, it was heated to 112° C., from which a very small amount of water was removed under reduced pressure. The addition of a propylene oxide monomer into the reactor was continued until pressure in the reactor was increased from vacuum to 0.5 kgf, and was then stopped. When the pressure of the reactor was reduced again to vacuum due to the activation of the double metal catalyst, addition of 573 g of propylene oxide at 1 g/min into the reactor was performed with control of a temperature of the reactor. Thereby, the pressure of the reactor was maintained at a predetermined level. After completion of the reaction, the reactor was allowed to stand at 112° C. for 30 min under vacuum to remove unreactant propylene oxide from a reaction product.

Thus, obtained polyol was analyzed with C-NMR to confirm a mol ratio of methine carbon. The results are shown in Table 5, below.

TABLE 5

Reaction Condition

| | | Number of | | | |
|---|---|---|---|---|---|
| | | Functional | Mol %* | | |
| C. Ex. No. | Catalyst | Groups | mm | mr + rm | rr |
| 1 | KOH | 3 | 27.2 | 38.8 | 34.0 |
| 2 | KOH | 2 | 28.5 | 39.2 | 32.3 |

*C-NMR Peak Area % (mm = 74.95, mr + rm. = 74.79, rr = 74.62)

### COMPARATIVE EXAMPLE 3

To assay properties of polyurethanes each composed of general polyol and stereoregular polyol, toluene diamine and catalyst were reacted, thus producing polyurethane foams each having a density of 17 and 27 kg/m³.

Further, the polyol prepared in Example 1 and the polyol prepared in Comparative Example 1 were used and produced into polyurethane compositions under the same conditions. Each composition was formed into polyurethane foams, which were then measured for physical properties. The results are summarized in Table 6, below.

TABLE 6

| | | Ex. 1 | C. Ex. 1 | Ex. 1 | C. Ex. 1 |
|---|---|---|---|---|---|
| Composition | Polyol | 70 | 70 | 100 | 100 |
| For | Polymer Polyol(POP) | 30 | 30 | — | — |
| Production | Water | 4.8 | 4.8 | 3.7 | 3.7 |
| of | Triethanolamine | 0.11 | 0.11 | 0.14 | 0.14 |
| Polyurethane | Silicone Surfactant | 1.3 | 1.3 | 1.2 | 1.2 |
| Foam (kg) | Stannous Octoate) | −0.28 | 0.28 | 0.26 | 0.26 |
| | Methylene Chloride | 8 | 8 | — | — |
| | Isocyanate (TDI-80) | 61.38 | 61.38 | 53.34 | 53.34 |
| Physical | Density (kg/m³) | 17.6 | 17.5 | 27.5 | 27.5 |
| Properties of | 25% Compressive | 16.0 | 15.4 | 25.4 | 24.4 |
| Polyurethane | Strength (25% 1LD) | | | | |
| Foam | (kg/314 cm³) | | | | |
| | 65% Compressive | 31.2 | 30.6 | 49.5 | 47.7 |
| | Strength (65% 1LD) | | | | |
| | (kg/314 cm³) | | | | |
| | Tear Strength (kg/cm²) | 0.44 | 0.42 | 0.42 | 0.41 |
| | Tensile Strength | 0.82 | 0.79 | 0.81 | 0.80 |
| | (kg/cm²) | | | | |
| | Elongation (%) | 92 | 90 | 70 | 71 |
| | Air Entraining | 3.31 | 3.2 | 1.65 | 1.61 |
| | (ft3/min) | | | | |
| | Resilience (%) | 34 | 33 | 41 | 41 |

From Table 6, it can be seen that the polyurethane foam resulting from the polyol of Example 1 is superior in almost physical properties, including compressive strength, tensile strength, elongation, etc., to those of the polyurethane foam prepared by using the polyol of Comparative Example 1.

As described hereinbefore, the present invention provides polyetherpolyol having stereoregularity and a preparation method thereof. The polyetherpolyol of the present invention can be applied to produce polyurethane having higher tensile strength, elasticity, elongation and so on, compared to polyurethanes resulting from atactic polyetherpolyol.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

We claim:

1. A method of preparing polyetherpolyol, comprising polymerizing an epoxy compound in a presence of a double metal cyanide complex catalyst represented by Formula 1, below:
Mₐ[M′(CN)₆]_{b}L_{c}L′_{d} Formula 1
wherein, M is Zn and M′ is Co (III), and L is tertbutanol, and L′ is polytetrahydrofuran, and a, b, c and d are integers, and a sum of a, b, c and d is equal to that of electrovalences of M and M′.

2. The method as defined in claim 1, wherein the epoxy compound comprises propylene oxide.
